(19) United States
(12) Patent Application Publication
(10) Pub. No.: US 2025/0064583 A1
(43) Pub. Date: Feb. 27, 2025

(54) MODULAR BIOPROSTHETIC VALVES AND METHODS FOR DESIGNING AND MAKING THEM

(71) Applicant: The Board of Trustees of the Leland Stanford Junior University, Stanford, CA (US)

(72) Inventors: Pearly Pandya, Stanford, CA (US); Matthew Park, Piedmont, CA (US); Hanjay Wang, Stanford, CA (US); Yuanjia Zhu, Stanford, CA (US); Y. Joseph Woo, Stanford, CA (US)

(21) Appl. No.: 18/940,688

(22) Filed: Nov. 7, 2024

Related U.S. Application Data
(63) Continuation of application No. PCT/US23/21416, filed on May 8, 2023.
(60) Provisional application No. 63/339,492, filed on May 8, 2022.

(51) Int. Cl. A61F 2/24

(52) U.S. Cl. CPC A61F 2/2418; A61F 2/2415; A61F 2/2448; A61F 2/2457; A61F 2250/006

(57) ABSTRACT

Modular prosthetic valves are disclosed that may support a customized number of leaflets. e.g., four or more leaflets carried by a support structure, such as a frame or stent, which may be used to replace diseased mitral, aortic, tricuspid, or pulmonary valves.

### STATEMENT REGARDING FEDERALLY SPONSORED RESEARCH AND DEVELOPMENT

None.

### RELATED APPLICATION DATA

The present application is a continuation of co-pending International Application No. PCT/US2023/021416, filed May 8, 2023, which claims benefit of U.S. provisional application Ser. No. 63/339,492, filed May 8, 2022, the entire disclosures of which are expressly incorporated by reference herein.

### TECHNCIAL FIELD

The present application relates to medical devices and, more particularly, to prosthetic valves, e.g., prosthetic heart valves including a customized number of leaflets and to methods for designing and making such valves.

### BACKGROUND

Structural valve degradation (SVD) is a major cause of limited durability of bioprosthetic valves and consists of any intrinsic permanent damage, such as calcification, leaflet fibrosis, and tears. Importantly, SVD is not equivalent at aortic and mitral positions: the rate of SVD is 19% in the aortic position and 34% in the mitral position. SVD of mitral bioprostheses is also characterized by an earlier onset and is overall less robustly studied. Due to the increased mechanical stress with higher closing pressures, cusp tears have been clinically observed at a higher prevalence with mitral bioprostheses.

Therefore, there is a need for improved prosthetic valves, e.g., that have improved prosthetic valve performance and/or may substantially mitigate structural valve degradation.

### SUMMARY

The present application is directed to medical devices and, more particularly, to prosthetic valves, e.g., prosthetic heart valves including a customized number of leaflets, e.g., four or more leaflets, and to methods for designing and making such valves.

When compared to aortic bioprostheses, structural valve degeneration of mitral bioprostheses is characterized by earlier onset, higher prevalence, and is less robustly studied. Currently available bioprosthetic mitral valves feature a tri-leaflet design, while the native mitral valve (“MV”) has two asymmetric leaflets. One aspect of the devices and methods herein is to use in silico analysis to optimize leaflet stresses and geometric orifice area (“GOA”) by varying the number of leaflets in a bioprosthetic mitral valve.

While all commercially available bioprosthetic valves include three symmetric leaflets (xenograft or polymeric), the modular bioprosthetic valves described herein allow for a customized number of leaflets carried by a frame, stent, or other implantable structure. The resulting valve assembly can support a plurality of leaflets, e.g., bi-leaflet, tri-leaflet, quadri-leaflet, or five or more leaflets; these leaflets may be symmetrical or asymmetrical depending on the valve position as well as patient-specific factors. The frame (e.g., for transcatheter application) or stent (e.g., open-heart application) may be interchanged during assembly. The frame/stent may be circular, D-shaped, or saddle-shaped, depending on pre-operative factors such as planarity of patient's native valve. The modular design may also support chordal attachment, e.g., if the replacement valve is intended to replace a tricuspid or mitral valve. The frame/stent may be made from, but not limited to, 3D printed biocompatible resin. The frame/stent may be assembled to include any combination of components, e.g., a sewing ring, cloth for preventing perivalvular leak, wire form, base frame, and/or leaflets (e.g., which may be bio-printed, xenograft, or polymeric), and/or may interface with respective implantation tools, such as valve attachment holders. Not all components must be utilized in all final prosthetic valves; for example, if only one native leaflet is being replaced within a surgical valve reconstruction, then the surgeon may choose to only use the stent component needed for the specific procedure.

Varying the number of leaflets may alter the leaflet stresses and/or orifice area, depending on the design chosen. Increasing the number of leaflets may be associated with improved bioprosthetic valve performance and/or may substantially mitigate structural valve degradation. Standard tri-leaflet valve designs may not be optimal for all valve positions, and so varying the number of leaflets may allow for more durable MV replacement devices and/or valve reconstruction techniques.

In accordance with one example, a prosthetic heart valve is provided for implantation at a valve annulus that includes an annular support structure; and a plurality of at least two leaflets attached to the support structure such that the leaflets open and close about a coaptation location.

In accordance with another example, a prosthetic heart valve is provided for implantation at a valve annulus that includes an annular support structure; and a plurality of at least four leaflets attached to the support structure such that the leaflets open and close about a coaptation location.

In accordance with yet another example, a prosthetic heart valve is provided for implantation at a valve annulus that includes an annular support structure; and a plurality of at least six leaflets attached to the support structure such that the leaflets open and close about a coaptation location.

In accordance with still another example, a method is provided for making a customized prosthetic valve for implantation with a subject's heart, the method including selecting a desired number of leaflets for the bioprosthetic valve based on the configuration of the valve annulus of the subject's heart; forming a support structure configured for implantation with the valve annulus; forming the number of leaflets; and attaching the leaflets to the support structure.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 shows an example of a prosthetic valve that includes a stent supporting four leaflets

FIG. 2A shows examples of a quadri-leaflet and six-leaflet prosthetic valve compared with a standard tri-leaflet valve using finite element analysis, which demonstrates a reduction in maximum von Mises stresses (units in Pa) mid-systole as the number of leaflets increases.

FIG. 2B shows an example of a bi-leaflet prosthetic valve in various positions during a cardiac cycle.

FIGS. 2C-2E are graphs showing evaluation of prosthetic valves including a plurality of leaflets. FIG. 2C shows maximum von Mises stresses of valves including between two and six leaflets (and projected for up to ten leaflets) mid-systole; FIG. 2D shows geometric orifice areas (“GOA”) of the valves mid-diastole; and FIG. 2E shows central leakage (as a percentage of orifice area) mid-systole for the valves.

FIG. 3 shows an example of a stent for a bi-leaflet prosthetic valve. In one example, the stent may be formed using biocompatible polyurethane RPU70 resin in a Carbon 3D printer. Similar stents may be printed or otherwise formed to support a variety of number of leaflets and, if desired, the stents may be interchanged to allow for implantation of a specific number of leaflets, e.g., depending on valve position and/or patient profile.

FIGS. 4A and 4B show examples of prosthetic valves including four leaflets. In FIG. 4A, all of the leaflets of the valve are symmetrical and have substantially equal surface areas. In FIG. 4B, the valve includes two pairs of leaflets with the first pair having substantially greater surface area than the second pair.

FIG. 5 shows yet another example of a prosthetic valve including four leaflets. In this example, the four leaflets are symmetrical and have been injection molded with silicone. Leaflets may also be casted or 3D printed, bio-printed, or sourced from xenograft tissue.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

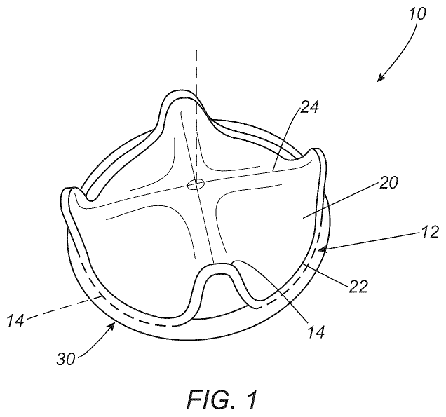

Turning to the drawings, FIG. 1 shows an example of a prosthetic heart valve 10 including a stent or frame 12 carrying a plurality of leaflets 20, e.g., four leaflets, and a sewing ring or cuff 30. The stent 12 generally is an annular ring or other support including a plurality of commissures 14, e.g., spaced apart circumferentially from one another, for supporting the leaflets 20. For example, the stent 12 may include circumferential regions 16 lying generally within a plane with the commissures 14 extending out of the plane, e.g., generally along an axis 18 normal to the plane. Alternatively, the circumferential regions may undulate or otherwise extend out of the plane if desired, e.g., including one or more lobes or scallops, and/or may have a non-circular shape, e.g., a generally “D” shape, a tri-lobal shape, a saddle shape, and/or other non-planar, three-dimensional shape, e.g., corresponding to the shape of a native valve annulus (not shown) within which the valve may be implanted.

In one example, the stent 12 is a single continuous member including the commissures 14 and circumferential regions 16, e.g., formed biocompatible materials, such as plastic, e.g., polyurethane, metal, e.g., stainless steel or Nitinol, or composite materials. The stent 12 may be formed by one or more of 3D printing, molding, casting, machining, and the like. Alternatively, the stent 12 may include a continuous ring extending around the circumference of the valve 10 and separate commissures may be attached to the ring (not shown). In a further alternative, a mesh stent, frame, or other annular support structure may be provided for carrying the leaflets 20 and supporting the valve when implanted within a native valve annulus such that the leaflets 20 may open and close, as desired.

The circumferential regions 16 of the stent 12 may be substantially rigid, e.g., to dilate a native valve annulus into which the valve 10 is implanted and/or the stent 12 may have variable rigidity around its circumference. In addition or alternatively, the commissures 14 may be substantially rigid and/or may be semi-rigid or flexible, e.g., to accommodate opening and closing of the leaflets 20. Alternatively, the stent 12 may be compressible, e.g., into a contracted configuration to facilitate introduction into a patient's body, for example, using a trans-catheter approach, and expandable, e.g., into an enlarged configuration within the native valve annulus. For example, the stent 12 may be biased to the enlarged configuration but may be compressed inwardly to the contracted configuration to accommodate introduction through a catheter, access port, and the like. In another alternative, the stent 12 may be provided in the contracted configuration without the material under stress and may be plastically deformed to direct the stent 12 to the enlarged configuration, e.g., using a balloon or other expandable member (not shown) that may be positioned within the stent 12 during implantation.

The leaflets 20 may be formed separately and attached to the stent 12, e.g., including outer edges 22 that extend between adjacent commissures 14, and inner, free edges 24 that may coapt with free edges of adjacent leaflets 20 to accommodate opening and closing of the valve 10. In one example, the leaflets 20 are formed individually from biological materials, e.g., bovine pericardium or other tissue, and attached to the stent 12 by one or more of suturing, bonding with adhesives, fusing, and the like. Optionally, a support frame, e.g., a “C” shaped strut (not shown) may be attached to the outer edges 22 of the leaflets 20, which may, in turn, be attached to the commissures 16 and/or other regions of the stent 12 to support the leaflets 20. Alternatively, the leaflets 20 may be bio-printed from biological material or may be formed from biocompatible synthetic materials, e.g., elastomeric material, such as silicone, or other plastic or composite material, e.g., formed by one or more 3D-printing, molding, casting, machining, and the like.

In a further alternative, a set of leaflets may be formed together, e.g., such that outer edges of the leaflets are attached together, while providing free edges that may open and close together. The leaflet assembly may then be attached to a stent or frame. For example, FIG. 5 shows an example of a set of four leaflets 120 formed from silicone, and an annular stent 112 formed from biocompatible polyurethane resin, e.g., formed by 3D-printing. The leaflet assembly may be attached to the stent 112, e.g., at commissures 114 and/or around the circumferential regions 116, for example, by suturing (not shown).

Returning to FIG. 1, a sewing cuff 30 may be permanently attached around an outer perimeter of the stent 12, e.g., by one more of suturing, bonding with adhesives, fusing, 10 interference fit, one or more connectors, and the like. The sewing cuff 30 may be formed from biocompatible materials, e.g., fabric, such as polyester (e.g., Dacron) and/or other synthetic material. The sewing cuff 30 may include multiple layers of fabric or may be filled with foam, fabric, and/or other material, which may be expandable or non-expandable, to facilitate securing the sewing cuff 30 within or adjacent a native valve annulus (not shown) and/or to facilitate 15 tissue ingrowth.

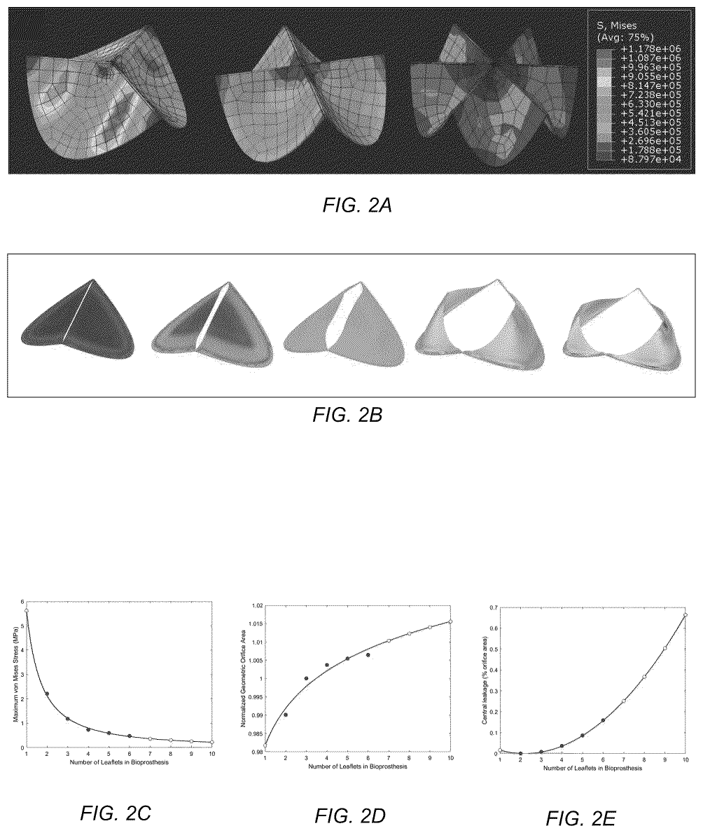

FIG. 2A shows examples of bioprosthetic heart valves including four and six leaflets. Generally, the customized bioprosthetic valves herein include a plurality of leaflets carried by a support structure, e.g., a stent or frame, that allows the leaflets to open and close about a coaptation location. Although FIG. 2A shows an example of a quadri-leaflet and six leaflet valve, it will be appreciated that any desired number of leaflets may be selected depending on the native anatomy and/or condition of the patient, e.g., two, three, four, five, six, or more leaflets. Further, the stent or frame may include a number of commissures and/or other supports sufficient to support the corresponding number of leaflets. For example, to support a valve including six leaflets, as shown in FIG. 2A, the stent may include six commissures (not shown) 25 to support the outer edges of the leaflets, e.g., generally constructed similar to the stent 12 and commissures 14 shown in FIG. 1.

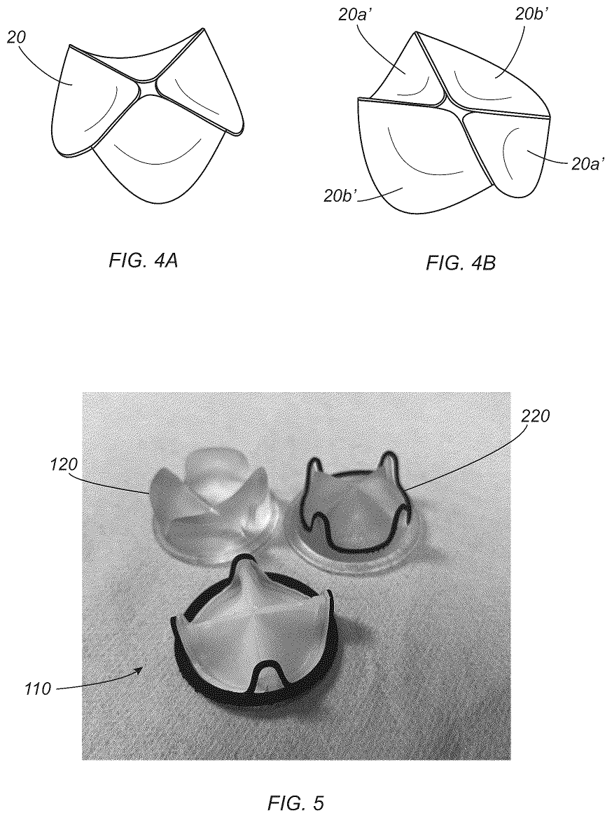

In one example, the leaflets of the valve may be substantially symmetrical and have surface areas that are substantially the same. For example, as shown in FIG. 4A, a set of four leaflets 20 are shown that are substantially identical to one another, e.g., having the same shape and surface area. Alternatively, if the valve includes even numbers of leaflets, the leaflets may be arranged in pairs opposite one another, with the opposite pairs having similar surface areas and/or other characteristics and one or more of the pairs having different sizes of characteristics than other pairs. For example, as shown in FIG. 4B, a set with four leaflets 20′ is shown where the leaflets include a first pair of leaflets 20a′ having a first surface area and a second pair of leaflets 40b′ having a second surface are greater than the first surface area.

In a further alternative, the valve may include a single leaflet or multiple desired leaflets that have a different surface area or other characteristics than the remaining leaflets (not shown). For example, if the valve includes four leaflets, three of the leaflets may have surface areas that are substantially the same and a fourth leaflet may have a surface area different than the three leaflets.

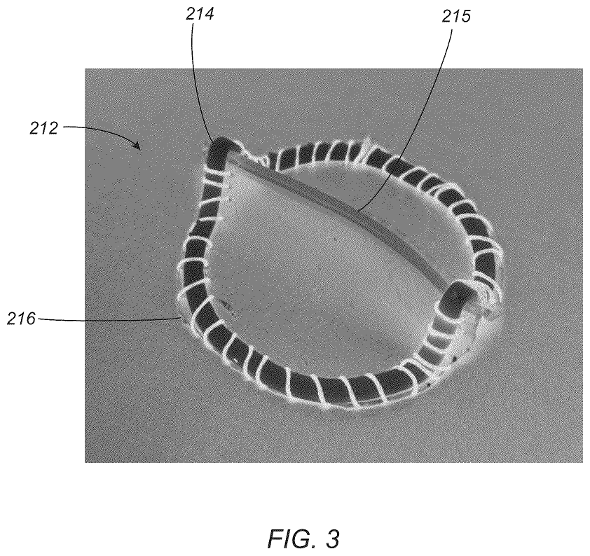

Turning to FIG. 3, another example of a support structure, i.e., stent 212 is shown, which may be formed, e.g., by 3D printing, molding, casting, and the like, and to which the leaflets (not shown) may be attached. Generally, similar to other devices herein, the stent 212 includes an annular frame or stent including a plurality of commissures, e.g., a pair of commissures 214 opposite one another around circumferential regions 216 to support the leaflets attached to the support structure 212. Optionally, the support structure may include one or more additional supports, e.g., strut 215 extending between the commissures 214, which may further support the leaflets and/or stent 212.

In examples, the support structure 212 may have a substantially circular annular shape, an oval shape, or a generally D-shape, e.g., to mimic the natural mitral annulus. The support structure may have an annular shape lying substantially within a plane or may have a non-planar shape about its periphery, e.g., similar to a native mitral valve annulus, depending on the valve being replaced and/or the patient's native anatomy.

Optionally, a sewing cuff (not shown) may be attached to the support structure 212, similar to other devices herein, e.g., including fabric or other flexible material, around a periphery of an annular frame to facilitate implantation within or otherwise to the native valve annulus. Alternatively, the support structure may include a tubular stent, e.g., including a mesh or other open walled-structure, configured for delivery during a transcatheter procedure.

Optionally, fabric covering, e.g., Dacron or other cloth, may be applied over one or more surfaces of the support structure, e.g., to cover any exposed surfaces in the final valve (or any of the other devices herein).

With further reference to FIGS. 2A-2E, in experiments, examples of bioprosthetic mitral valves with varying number of leaflets were designed using Bézier curvature and quadratic spline geometry. Leaflets were modeled with standard mechanical parameters for fixed bovine pericardial tissue. A mesh of each design was structurally evaluated using finite element modeling software Abaqus CAE. Valve closure was simulated with 120 mmHg pressure loaded on the ventricular side of the leaflets (FIG. 2A); valve opening was simulated using a dynamic explicit finite element analysis with 0-8 mmHg pressures loaded on the atrial side. Pressure differential values were quantified through ex vivo hemodynamic testing of a stented bioprosthetic MV. Maximum von Mises stress, orifice area, and central leakage area were assessed for each design.

As shown in FIG. 2B, the number of leaflets (1-10) in the bioprosthetic MVs increased, there was a decrease in maximum von Mises stresses as well as an increase in orifice area, as shown in FIG. 2C. Compared to the standard tri-leaflet design, a quadri-leaflet (four leaflets) pattern may reduce stresses by 38% mid-systole and 18% mid-diastole and increase GOA by 1%. Stress concentrations within the leaflet can cause tearing or initiate calcification, so a reduction in leaflet stresses may mitigate effects of SVD. Reduction in orifice area caused by calcification has shown to create detrimental downstream flow patterns, so ensuring the new valve design maintains GOA is also crucial. As shown in FIG. 2D, for example, as the number of leaflets increase, uniform coaptation can become increasingly complex, resulting in a small area of leakage in the valve center: 0.01% of the orifice area may leak with the current tri-leaflet design, 0.04% with quadri-leaflet, and 0.16% with hexa-leaflet. Therefore, a quadri-leaflet pattern may optimize GOA and decrease leaflet stresses, while limiting central leakage.

Increasing the number of leaflets may be associated with improved bioprosthetic valve performance. These findings suggest that the standard tri-leaflet design may not be optimal for all valve positions and varying the number of leaflets may allow for more durable MV replacement devices and valve reconstruction techniques. In particular, a quadri-leaflet pattern may optimize leaflet stresses and GOA, while limiting central leakage.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A prosthetic heart valve for implantation at a valve annulus, comprising:
an annular support structure; and
a plurality of at least two leaflets attached to the support structure such that the leaflets open and close about a coaptation location.

2. The valve of claim 1, wherein the leaflets are substantially symmetrical and have surface areas that are substantially the same.

3. The valve of claim 1, wherein the valve includes four leaflets and wherein the leaflets include a first pair of leaflets having a first surface area and a second pair of leaflets having a second surface are greater than the first surface area.

4. The valve of claim 1, wherein the valve includes four leaflets and wherein the leaflets include three leaflets having surface areas that are substantially the same and a fourth leaflet that has a surface area different than the three leaflets.

5. The valve of claim 1, wherein the support structure comprises a frame or stent having a substantially circular shape.

6. The valve of claim 1, wherein the support structure comprises a frame or stent having a generally D-shape.

7. The valve of claim 1, wherein the support structure has an annular shape lying substantially within a plane.

8. The valve of claim 1, wherein the support structure has a non-planar shape about its periphery.

9. The valve of claim 1, wherein the support structure has a non-planar shape similar to a native mitral valve annulus.

10. The valve of claim 1, further comprising a suture ring attached to the support structure.

11. The valve of claim 1, wherein the support structure comprises one of a frame or stent formed from synthetic material.

12. The valve of claim 1, wherein the support structure comprises a stent configured for delivery during a transcatheter procedure.

13. The valve of claim 1, further comprising fabric covering exposed surfaces of the support structure.

14. The valve of claim 1, wherein the leaflets are formed from synthetic, polymeric material.

15. The valve of claim 1, wherein the leaflets are formed from biological material.

16-17. (canceled)

18. The valve of claim 1, wherein the support structure comprises one or more features for chordal attachment.

19. The valve of claim 1, wherein the number of leaflets is at least six.

20. The valve of claim 1, wherein the number of leaflets is at least five.

21. A prosthetic heart valve for implantation at a valve annulus, comprising:
an annular support structure; and
a plurality of at least four leaflets attached to the support structure such that the leaflets open and close about a coaptation location.

22-41. (canceled)

42. A method for making a customized prosthetic valve for implantation with a subject's heart, comprising:
selecting a desired number of leaflets for the bioprosthetic valve based on the configuration of the valve annulus of the subject's heart;
forming a support structure configured for implantation with the valve annulus;
forming the number of leaflets; and
attaching the leaflets to the support structure.

43-53. (canceled)
